(19) United States
(12) Patent Application Publication
(10) Pub. No.: US 2025/0018385 A1
(43) Pub. Date: Jan. 16, 2025

(54) HOLLOW NEEDLE FOR A SAMPLE PIPETTOR

(71) Applicant: TECAN TRADING AG, Mannedorf (CH)

(72) Inventors: Roland WURGLER, Mannedorf (CH); Peter KUMMERLI, Stettlen (CH); Dominic ERB, Winterthur (CH); Paul TANASESCU, Zurich (CH)

(21) Appl. No.: 18/897,942

(22) Filed: Sep. 26, 2024

Related U.S. Application Data
(62) Division of application No. 16/783,368, filed on Feb. 6, 2020.

(30) Foreign Application Priority Data
Feb. 12, 2019 (EP) 19156762.7

(51) Int. Cl. B01L 3/02

(52) U.S. Cl. CPC B01L 3/021; B01L 2300/0672

(57) ABSTRACT

A hollow needle for piercing a closure of a sample vessel, wherein the hollow needle has a cylindrical hollow profile extending along a longitudinal axis and having a tip at a first end thereof, where the hollow needle has a first section that has a tip and a second section that has a larger diameter than the first section, and a transition between the first section and the second section has at least one cutting edge that extends from the first section to the second section.

### CROSS-REFERENCE TO RELATED APPLICATION

This application is a divisional application of U.S. patent application Ser. No. 16/783,368, filed on Feb. 6, 2020, which, in turn, claims priority to European Patent Application No. 19156762.7 filed on Feb. 19, 2019, the contents of each is incorporated by reference in its entirety.

### FIELD OF THE INVENTION

The present invention relates to a hollow needle for a sample pipettor in a device for laboratory automation, in particular for taking sample liquid from a sealed sample vessel, wherein for taking the sample liquid the seal of the sample vessel is pierced with the hollow needle. Sample liquids can also be dispensed into sample containers with a cap, wherein the hollow needle pierces the cap before dispensing the sample liquid. A device for laboratory automation can be a pipetting robot or an autosampler for chromatographs or spectrometers, for example. Sample vessel can be defined as a container that contains analytes, solutions of analytes or biological fluids, or that contains reagents. A closure can be understood to be a closure with a built-in plastic or rubber membrane or a plastic or rubber stopper.

### DESCRIPTION OF THE PRIOR ART

Hollow needles are known from the prior art, which for piercing the closure of a sample vessel comprises a cylindrical hollow profile with a first section and with a second section. The first section includes the tip of the hollow needle and the second section has a larger diameter than the first section. A conical region connects the first section to the second section. The tip of the hollow needle includes a cutting edge with which the closure is cut across the width of the first section.

This has the disadvantage that the required piercing force increases as soon as the second section with the larger diameter is to be pushed through the closure. When pulling out, the clamping forces acting on the hollow needle through the closure can be so great that the closure is pulled off the sample vessel or that the closure is pulled out of the sample holder together with the sample vessel. The increased puncture force also puts a strain on the mechanics of the device for laboratory automation and leads to premature wear and tear. When piercing the closure, it must also be ensured that the closure is only pierced and that no material of the closure is punched out. The punched material can either clog the needle or contaminate the sample, both of which should be avoided at all costs.

### SUMMARY OF THE INVENTION

It is an object of the present invention to provide a hollow needle in which the clamping forces are as small as possible when piercing or passing through a closure.

This object is solved by a hollow needle with the features of claim 1. Other embodiments of the hollow needle are defined by features of further claims.

A hollow needle according to the invention for piercing a closure of a sample vessel comprises a cylindrical hollow profile extending along a longitudinal axis and having a tip at its first end. The hollow needle has a first section that encompasses the tip and a second section that is larger in diameter than the first section. A transition between the first section and the second section comprises at least one cutting edge extending from the first section to the second section. The cutting edge means that when the closure is pierced, the closure is further cut open by the cutting edge, which reduces the clamping forces acting laterally on the hollow needle.

In one embodiment, the transition comprises two or more cutting edges extending from the first section to the second section.

In one embodiment, the cutting edges are evenly distributed around the circumference of the transition. For example, two cutting edges can be provided on the circumference opposite each other or three cutting edges can be provided, which are offset to each other by an angle of 120° with respect to the longitudinal axis. In principle, any number of cutting edges can be arranged at any angle to each other around the circumference of the transition.

In one embodiment, the cutting edges extend substantially perpendicular to the longitudinal axis or the cutting edges extend at an angle of less than 90° to the longitudinal axis. A cutting edge aligned perpendicular to the longitudinal axis is easy to produce and a cutting edge at an angle to the longitudinal axis causes a continuous increase in the cutting length as the transition between the first section and the second section of the hollow needle is inserted into the closure.

In one embodiment, each cutting edge is formed by two flanks adjacent thereto, wherein the two adjacent flanks extend at the same angle to the longitudinal axis or wherein the two adjacent flanks extend at different angles to the longitudinal axis. For example, the first flank extends at a first angle and the second flank extends at a second angle.

The two flank angles can be matched to the angles of the surfaces of the tips. For a tip with asymmetrically angled surfaces, the first surface of the tip is aligned at a first surface angle. The surface of the tip opposite the first surface or the resulting intersection edge of two adjacent surfaces of the tip opposite the first surface is arranged at an edge angle smaller than the surface angle. Accordingly, the angle of the first flank, which is aligned with the first surface of the tip in the direction of the longitudinal axis, is smaller than the angle of the second flank, which is aligned with the second surface or the edge of the tip. For example, if the first surface angle of the tip is 15° and the opposite edge angle is 4°, the first flank angle can be 4° and the second flank angle 15°. Accordingly, when the needle tip is inserted into the closure, the needle is pushed to a first side. When the transition is inserted into the closure, the needle is pushed to the side opposite the first side. Accordingly, the asymmetrical load on the needle is compensated, resulting in less or no deflection of the needle. With symmetrical needle tips, the flanks of the cutting edge can also be formed symmetrically.

In one embodiment, the tip comprises a first surface which extends at an angle to the longitudinal axis over substantially the entire cross-section. This design is easy to manufacture, but on the side of the first surface, greater radial forces act on the tip of the hollow needle when the hollow needle tip is inserted into a closure of a sample vessel. Alternatively, the tip comprises a first surface and a second surface extending at an angle to the longitudinal axis over substantially half of the cross-section. Due to the symmetrical design of the tip in relation to the longitudinal center axis of the hollow needle, the radial forces acting on the hollow needle tip during insertion into the closure are symmetrical in relation to the longitudinal center axis.

In one embodiment, the flanks of the cutting edge are aligned along the longitudinal axis with the surfaces of the tip.

In one embodiment, a first flank angle of the cutting edge substantially corresponds to an edge angle of the tip or a second surface angle of the tip, and wherein a second flank angle of the cutting edge substantially corresponds to a first surface angle of the tip.

In one embodiment, the tip has an opening provided in the first surface or the tip has an opening provided adjacent to the first surface.

In one embodiment, the second section has at least one recess which extends substantially along the longitudinal axis over at least a portion of the second section. The at least one recess limits a passage channel through the closure in the case of a hollow needle pushed through the closure and allows the interior of the sample vessel to be connected to the environment for pressure equalization during the aspiration or ejection of liquid into or out of the hollow needle.

In one embodiment, two or more recesses are provided in the second section, which are evenly distributed around the circumference of the second section. For example, two, three, four, five, six or more recesses can be evenly distributed around the circumference. An irregular arrangement of any number of recesses would also be possible.

In one embodiment, the hollow needle comprises a third section which adjoins the second section on the side of the second section opposite the first section. The third section may have a larger diameter than the second section. However, the diameter can also be the same size. The transition between the second section and the third section can be step-shaped or continuous.

In one embodiment, the third section includes a stop which extends beyond the diameter of the second section and which is spaced from a second end opposite the first end of the hollow needle. The third section may further include a cone, which is formed at the second end.

In one embodiment, the hollow needle comprises an inner tube and an outer tube. The inner tube extends over the entire length of the hollow needle and the tip is formed in the inner tube. The outer tube extends around the inner tube at least over a section of the second section. The inner dimensions of the outer tube are matched to the outer dimensions of the inner tube in such a way that the outer tube can be pushed onto the inner tube. The outer tube can be firmly connected to the inner tube using any joining method, for example laser welding, TIG welding or brazing.

In one embodiment, at least one cutting edge is formed in the outer tube. The cutting edge can be made before the outer tube is pushed onto the inner tube, which makes it much easier to produce the cutting edge.

In one embodiment, the hollow needle comprises a connecting tube which extends around the inner tube from the outer tube to the second end of the hollow needle. The inner dimensions of the connecting tube are matched to the outer dimensions of the inner tube in such a way that the connecting tube can be pushed onto the inner tube. The connecting tube can be permanently connected to the inner tube by any joining method, for example laser welding, TIG welding or brazing.

In one embodiment, the cone, which is formed at the second end of the hollow needle, is formed in the connecting tube.

In one embodiment, the hollow needle includes a sleeve-shaped stop that extends around the inner tube over an area of the third section. The internal dimensions of the stop are matched to the external dimensions of the connecting tube in such a way that the stop can be pushed onto the connecting tube. The stop can be permanently connected to the connecting tube by any joining method, for example laser welding, TIG welding or brazing.

In one embodiment, the inner tube, the outer tube and the connecting tube comprise a first material and the stop comprises a second material. Alternatively, all these components may comprise the same or different materials. For example, both materials are CrNi steels. For example, the first material is X2CrNiMo17-12-2 and the second material is X8CrNiS18-9.

For example, the inner tube has an inner diameter of 0.5 mm, an outer diameter of 0.8 mm and a length of 155 mm. For example, the inner diameter can be in a range of 0.3 to 0.7 mm and the outer diameter in a range of 0.6 to 0.8 mm. For example, the outer tube has an inner diameter of 0.8 mm, an outer diameter of 1.6 mm and a length of 111 mm. For example, the inner diameter can be in a range of 0.6 to 0.8 mm and the outer diameter in a range of 1.4 to 2.0 mm. For example, the connecting tube has an inner diameter of 0.8 mm, an outer diameter of 2 mm and a length of 34 mm. For example, the stop has an inner diameter of 2 mm, an outer diameter of 4 mm and a length of 4 mm. For example, the first flank of the cutting edge of the transition is aligned at an angle of 15° with respect to the longitudinal axis and the second flank is aligned at an angle of 4°. For example, the length of the recesses in the outer tube is 83 mm and the width and depth is 0.3 mm. For example, for the tip of an asymmetrical first embodiment, the surface angle at which the first surface of the tip is aligned with respect to the longitudinal axis is 15° and the edge angle of the third edge opposite the first surface is 4°. For example, the surface angle can be 10° to 20° and the edge angle 2° to 6°. For example, for the tip of a symmetrical second embodiment, the surface angle of the first surface and the second surface is 15°. The surface angles can be 10° to 20°, for example.

In one embodiment, the tip comprises at least one cutting edge and the at least one cutting edge of the transition is aligned with the at least one cutting edge of the tip. If the tip has two or more cutting edges, the transition has the same number of cutting edges and each of the cutting edges of the transition is aligned with a corresponding cutting edge of the tip. Aligned means here that they are aligned with each other along the longitudinal axis.

The aforementioned embodiments of the hollow needle can be used in any combination, as long as they do not contradict each other.

### DESCRIPTION OF THE FIGURES

Embodiment examples of the present invention are explained in more detail below using Figs. These are for explanatory purposes only and are not to be interpreted restrictively, wherein:

FIG. 1 shows a side view of a hollow needle according to the invention;

FIG. 2 shows a sectional view through the hollow needle of FIG. 1;

FIG. 3A shows a view of detail V of FIG. 1;

FIG. 3B shows a view of detail X of FIG. 2;

FIG. 3C shows a rear view of FIG. 3A;

FIG. 4 shows a view of the detail Y of FIG. 2;

FIG. 5 shows a view of section B-B of FIG. 2;

FIG. 6A shows a view of the detail W of FIG. 3C;

FIG. 6B shows a detailed view of the tip of FIG. 3B;

FIG. 6C shows a rear view of FIG. 6A;

FIG. 7A shows a sectional view of a hollow needle according to the prior art;

FIG. 7B shows a sectional view of a hollow needle according to the invention;

FIG. 8A shows a detailed view of an alternative hollow needle tip;

FIG. 8B shows a sectional view of the hollow needle tip of FIG. 8A;

FIG. 8C shows a rear view of FIG. 8A;

FIG. 9A shows a sectional view of a hollow needle according to the prior art;

FIG. 9B shows a sectional view of a hollow needle according to the invention; and

FIG. 10 shows the steps for inserting a hollow needle according to the invention into a sample pipettor.

### DETAILED DESCRIPTION OF THE INVENTION

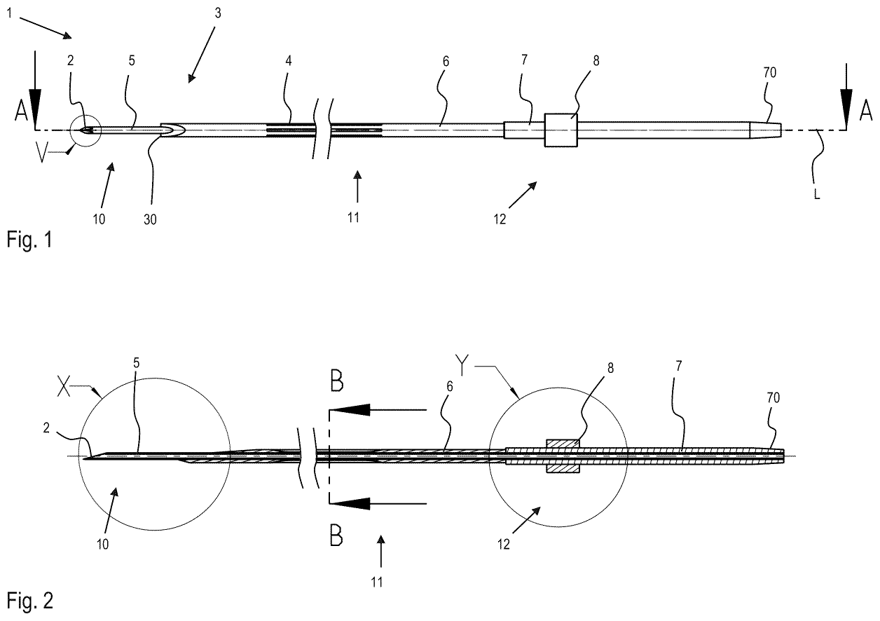

FIG. 1 shows a side view of a hollow needle according to the invention and FIG. 2 shows a sectional view through the hollow needle of FIG. 1. The hollow needle 1 comprises a cylindrical hollow profile which extends along a longitudinal axis L and at the first end of which a tip 2 is provided. The hollow profile can have a circular, oval or n-angular cross-section, wherein n is equal to or greater than or equal to three. The n-angular cross-section can be a regular or irregular n-angle. The hollow needle 1 has a first section 10, which includes the tip 2. The hollow needle 1 also includes a second section 11, which has a larger diameter than the first section 10. A transition 3 between the first section 10 and the second section 11 comprises at least one cutting edge 30 extending from the first section 10 to the second section 11. In the embodiment shown, the hollow needle 1 further comprises a third section 12 which adjoins the second section 11 and extends opposite the first end to the second end of the hollow needle. The length of the first section 10 is a multiple of the diameter of the first section 10 and the length of the second section 11 is a multiple of the diameter of the second section 11 and is a multiple of the length of the first section 10. The length of the third section 12 is a multiple of the length of the first section 10 and a fraction of the length of the second section 11. In a central area of the second section 11, several recesses 4 are evenly distributed around the circumference.

The end of recesses 4 directed towards the tip 2 is spaced from the cutting edge 30 and the end of recess 4 directed away from the tip 2 is spaced from the third section 12. The third section 12 has a diameter only slightly larger than the second section 11. The third section 12 comprises a connecting tube 7 which extends from the second section 11 to the second end of the hollow needle 1. In the third section 12 a cone 70 is formed at the second end of the hollow needle 1. The third section 12 includes a stop 8, the diameter of which is larger than that of the connecting tube 7, and the width of the stop 8 is essentially equal to its length. The distance between the stop 8 and the second end of the hollow needle 1 is several times the length of the stop 8. The hollow needle 1 comprises an inner tube 5 which extends from the first end, i.e. from the tip 2, to the second end, i.e. to the cone 70. The tip 2 surrounds the first end of the inner tube 5. The hollow needle 1 further comprises an outer tube 6, which extends in the second area 11 around and adjacent to the inner tube 5. The recesses 4 are formed in the outer tube 6. The hollow needle 1 further comprises a connecting tube 7, which extends in the third area 12 around the inner tube 5 and adjacent to it. The stop 8 is designed as a sleeve, which extends around the connecting tube 7 and lies against it. The inner tube 5, the outer tube, 6, the connecting tube 7 and the stop 8 are firmly connected to each other by laser welding.

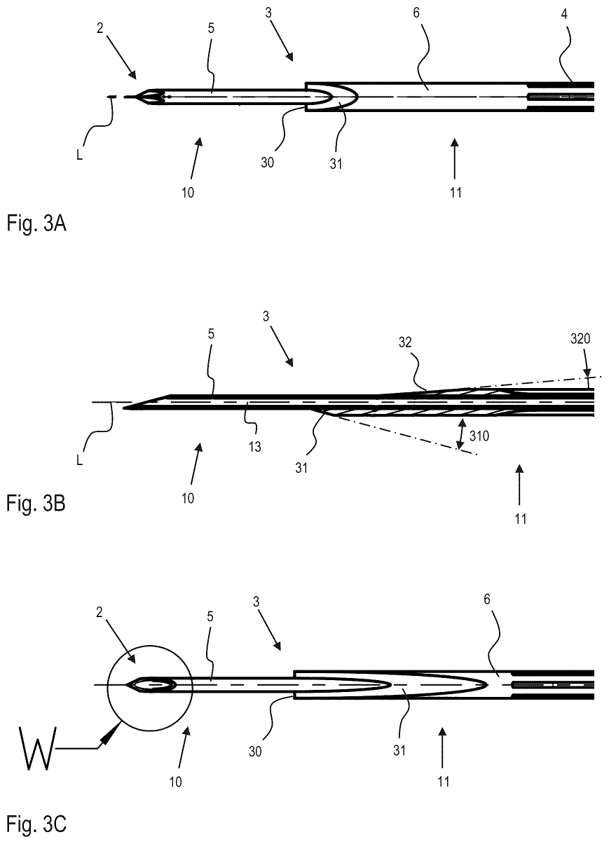

FIG. 3A shows a view of detail V of FIG. 1, FIG. 3B shows a view of detail X of FIG. 2 and FIG. 3C shows a rear view of FIG. 3A. The figure shows the first section 10 and a part of the second section 11 with a part of the recesses 4. The cutting edge 30 and a first flank 31 is visible of the transition 3. The cutting edge 30 extends essentially perpendicular to the longitudinal axis L of the hollow needle. The first flank 31 is produced by grinding the outer tube 6 at an angle before assembling it with the inner tube 5. In the interior of the inner tube 5, a hollow channel 13 extends over its entire length. The first flank 31 extends at a first angle 310 from the cutting edge 30 obliquely in the direction of the second end of the hollow needle 1. A second flank 32 extends at a second angle 320 from the cutting edge 30 obliquely in the direction of the second end of the hollow needle 1. In this illustration, the first angle 310 is a multiple of the second angle 320. The ends of the recesses 4 directed towards the transition 3 are spaced from the edge of the first and second flanks 31, 32 directed towards the second end of the hollow needle 1.

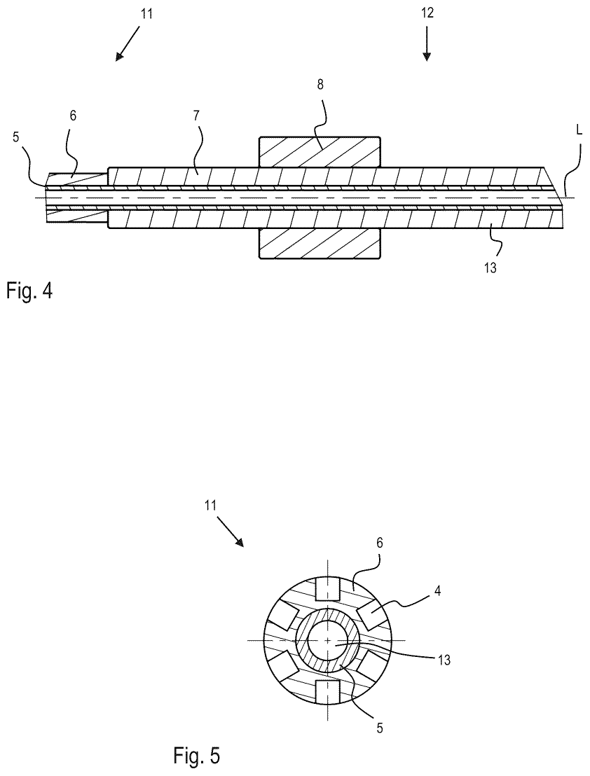

FIG. 4 shows a view of detail Y of FIG. 2. The inner tube 5 with the hollow channel 13 extends along the longitudinal axis L. The outer tube 6 surrounds the inner tube 5 in the second section 11 and the connecting tube 7 surrounds the inner tube 5 in the third section 12. The connecting tube 7 is flush with the outer tube 6. The stop 8 surrounds an area of the connecting tube 7. The wall thickness of the stop 8 is greater than the wall thickness of the connecting tube 7.

FIG. 5 shows a view of section B-B of FIG. 2, showing a section through the area of the recesses 4 in the second area 11 of the hollow needle 1. Six recesses 4 are evenly distributed around the circumference of the outer tube 6. The depth of the recesses 4 is greater than half the wall thickness of the outer tube 6, and the wall thickness of the outer tube 6 is a multiple of the wall thickness of the inner tube 5.

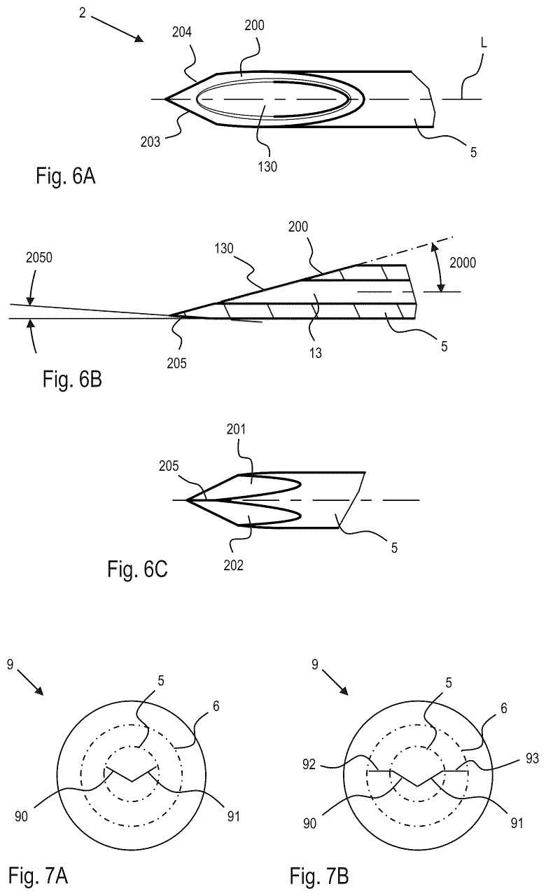

FIG. 6A shows a view of the detail W of FIG. 3C, FIG. 6B shows a detail view of the tip of FIG. 3B, and FIG. 6C shows a rear view of FIG. 6A. The tip 2 of hollow needle 1 comprises a first surface 200, which extends at a surface angle 2000 with respect to the longitudinal axis L. The first surface 200 can be created by grinding the inner tube 5.

On the opposite side of the first surface 200 with respect to the longitudinal axis L, a second surface 201 and a third surface 202 are provided, wherein the second surface 201 is arranged at an angle to the first surface 200 and the third surface 202. A first edge 203 is formed by the intersection line of the first surface 200 with the second surface 201, a second edge 204 is formed by the intersection line of the first surface 200 with the third surface 202, and a third edge 205 is formed by the intersection line of the second surface 201 with the third surface 203. The third edge 205 extends at an edge angle 2050 with respect to the longitudinal axis. When the tip 2 is inserted into the closure, first radial forces act on the hollow needle 1 from the side of the first surface 200. The design of the second surface 201, the third surface 202, together with the third edge 205, generate second radial forces in a direction opposite to the direction of the first forces. The tip 2 of the hollow needle 1 is accordingly less pushed to the side, allowing the hollow needle to be inserted in a more centered manner into a sample vessel. The hollow channel 13 extends through the entire inner tube 5 and opens into an opening 130, which is enclosed by the first surface 200.

FIG. 7A shows a sectional view 9 of a hollow needle according to the prior art, as is produced when a tip, as shown in FIGS. 6A to 6C, is pushed through a closure. A first section 90 is cut into the closure by the first cutting edge 203 and a second section 91 is cut into the closure by the second cutting edge 204. The outer ends of the first and second sections 90, 91 are at a diameter corresponding to that of the first section 10 and the inner tube 5 respectively. If the second section 11 or the outer tube 6 is to be pushed through the closure, sections 90, 91 must be widened, creating radial clamping forces which act on the hollow needle. The increased radial forces also increase the force required to push the hollow needle through the closure or to pull the hollow needle out of the closure.

FIG. 7B shows a sectional view of a hollow needle according to the invention, as it is produced when the tip is first pushed through the closure according to FIGS. 6A to 6C and then the transition 3 with two cutting edges 30 opposite each other on the circumference, as shown in FIG. 3A. The tip 2 again creates a first section 90 and a second section 91. When the transition 3 is pushed through the closure, the cutting edges 30 create a third section 92, which adjoins the first section 90, and a fourth section 93, which adjoins the second section 91. The outer ends of the third and fourth section 92, 93 are on a diameter corresponding to that of the second section 11 or the outer tube 6 respectively. When the transition 3 is pushed through the closure, its cutting edges 30 produce cutting forces, but these are less than the forces needed to expand the closure if there are no cutting edges 30. Because all sections 90, 91, 92, 93 together extend over essentially the diameter of the second section, the forces required to expand the closure are much smaller.

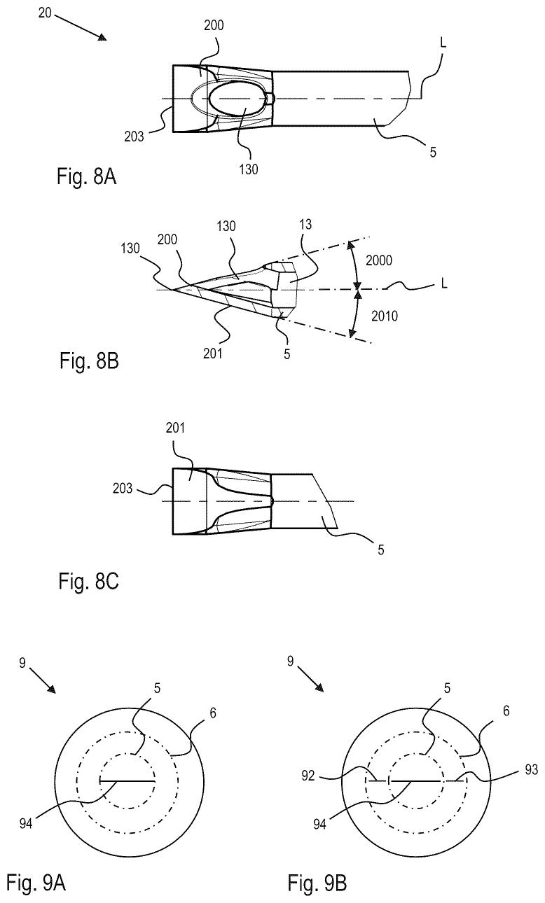

FIG. 8A shows a detailed view of an alternative hollow needle tip 20, FIG. 8B shows a sectional view of the hollow needle tip 20 of FIG. 8A and FIG. 8C shows a rear view of FIG. 8A. In this embodiment, the first section 10 or the inner tube 5 is compressed and then ground off from two sides symmetrically with respect to the longitudinal axis L. This results in a first surface 200 and a second surface 201. The first surface 200 is oriented at a first surface angle 2000 with respect to the longitudinal axis L and the second surface 201 is oriented at a second surface angle 2010. The intersection line of the first surface 200 with the second surface 201 results in the first edge 203. Alternatively, two first surfaces 200 and two second surfaces 201 could be ground to tip 20, wherein the two first surfaces 200 are oriented at an angle with respect to each other and wherein the two second surfaces 201 are oriented at an angle with respect to each other. This creates two first edges 203, which are oriented at an angle with respect to each other. A symmetrical arrangement and orientation of the first and second surfaces results in a symmetrical arrangement and orientation of the first edges. In the tip 20 shown, the hollow channel 13 opens into an opening 130, which is located adjacent to the first surface 200.

FIG. 9A shows a sectional view of a hollow needle according to the prior art. The tip 20 shown in FIGS. 8A to 8C cuts only a central fifth section 94 into the closure. The fifth section 94 extends essentially over the diameter of the first section 10, or inner tube 5. By compressing the inner tube 5, the tip 20 is widened at its end, causing the first cutting edge 203 to protrude over the diameter of the inner tube 5.

FIG. 9B shows a sectional view of a hollow needle according to the invention. Through the cutting edges 30 of transition 3, a third section 92 and a fourth section 93 are cut into the closure, wherein the third and fourth sections 92, 93 are laterally adjacent to and aligned with the central fifth section 94.

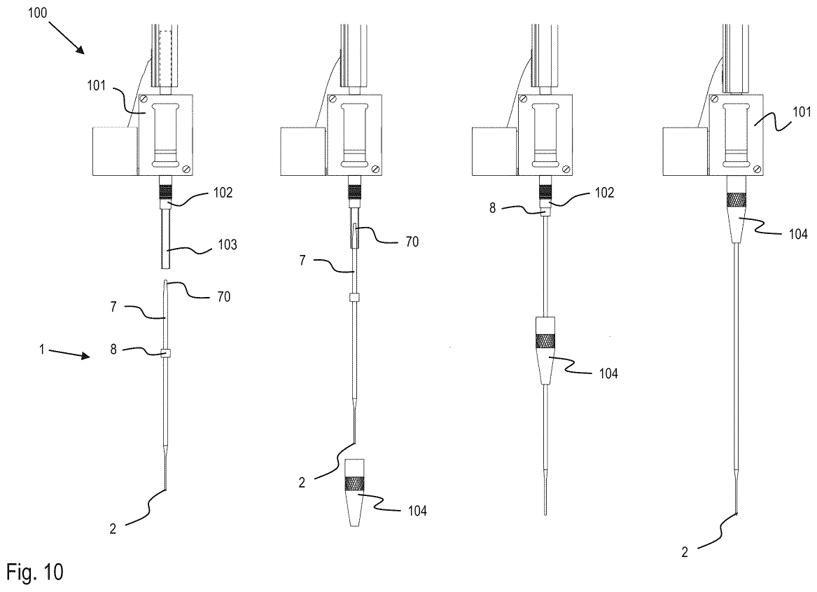

FIG. 10 shows the steps for inserting a hollow needle 1 according to the invention into a sample pipettor 100. The sample pipettor 100 comprises a holder 101 with a connecting sleeve 102, in which a tube 103 is slidably mounted, and a union nut 104, with which the hollow needle can be attached to the holder 101. For pipetting, negative or positive pressure can be generated in tube 103. The holder 101 can be moved horizontally and vertically. The connecting tube 7 of the hollow needle 1 is inserted with the cone 70 into the tube 103. The tube 103 is pushed together with the hollow needle 1 into the connecting sleeve 102 up to the stop 8. The union nut 104 is pushed from the tip 2 over the hollow needle 1 up to the stop 8 and the union nut 104 is screwed tight on the connecting sleeve 102.

LIST OF REFERENCE NUMERALS

| | |
|---|---|
| 1 | Hollow needle |
| 10 | First section |
| 11 | Second section |
| 12 | Third section |
| 13 | Hollow channel |
| 130 | Opening |
| 2 | Tip |
| 20 | Tip |
| 200 | First surface |
| 2000 | First surface angle |
| 201 | Second surface |
| 2010 | Second surface angle |
| 202 | Third surface |
| 203 | First edge |
| 204 | Second edge |
| 205 | Third edge |
| 2050 | Edge angle |
| 3 | Transition |
| 30 | Cutting edge |
| 31 | First flank |
| 310 | First angle |
| 32 | Second flank |
| 320 | Second angle |
| 4 | Recess |
| 5 | Inner tube |
| 6 | Outer tube |
| 7 | Connecting tube |
| 70 | Cone |
| 8 | Stop |
| 9 | Sectional view |
| 90 | First section |
| 91 | Second section |
| 92 | Third section |
| 93 | Fourth section |
| 94 | Fifth section |
| 100 | Pipettor |
| 101 | Holder |
| 102 | Connection |
| 103 | Tube |
| 104 | Union nut |
| L | Longitudinal axis |

What is claimed is:

1. A hollow needle for piercing a closure of a sample vessel, comprising:
an inner tube having a first outer diameter and which extends from a first end to a second end, the inner tube having a cylindrical hollow profile extending along a longitudinal axis thereof and having a tip at the first end thereof;
an outer tube having a second outer diameter that is larger than the first outer diameter and which extends around and adjacent to the inner tube, the outer tube having a first end offset from the first end of the inner tube and a second end, a portion from the first end of the inner tube to the first end of the outer tube forming a first section, a portion from the first end of the outer tube to the second end of the outer tube forming a second section that extends along the longitudinal axis of the inner tube; and
at least one cutting edge at a transition between the first section and the second section, the at least one cutting edge extending from the first outer diameter of the first section to the second outer diameter of the second section, wherein the at least one cutting edge comprises two surfaces formed in the outer tube.

2. The hollow needle according to claim 1, comprising two or more cutting edges extending from the first section to the second section.

3. The hollow needle according to claim 2, wherein the cutting edges are evenly distributed around a circumference at the transition.

4. The hollow needle according to claim 2, wherein the cutting edges extend substantially perpendicular to the longitudinal axis of the inner tube or wherein the cutting edges extend at an angle of less than 90° to the longitudinal axis of the inner tube.

5. The hollow needle according to claim 1, wherein the two surfaces extend at an equal angle to the longitudinal axis of the inner tube or the two surfaces extend at different angles to the longitudinal axis of the inner tube.

6. The hollow needle according to claim 1, wherein the tip comprises a first surface which extends at an angle to the longitudinal axis of the inner tube over substantially an entire cross-section of the inner tube.

7. The hollow needle according to claim 6, wherein the two surfaces of the cutting edge are aligned along the longitudinal axis of the inner tube with the first surface of the tip.

8. The hollow needle according to claim 7, wherein an angle of a first of the two surfaces of the cutting edge substantially corresponds to an edge angle of the tip or a second surface angle of the tip, and wherein an angle of a second of the two surfaces of the cutting edge substantially corresponds to a first surface angle of the tip.

9. The hollow needle according to claim 6, wherein the tip has an opening which is provided in the first surface or which is provided adjacent to the first surface.

10. The hollow needle according to claim 1, wherein at least one recess is provided in the outer tube which extends substantially along the longitudinal axis of the inner tube.

11. The hollow needle according to claim 10, wherein two or more recesses are provided in the outer tube which are evenly distributed around the circumference of the outer tube.

12. The hollow needle according to claim 1, comprising a connecting tube forming a third section extending outward from the second end of the outer tube along the longitudinal axis of the inner tube so that the third section adjoins the second section on the side of the second section opposite the first section.

13. The hollow needle according to claim 12, comprises a stop which projects beyond a diameter of the connecting tube and which is spaced from a second end opposite the first end of the inner tube.

14. The hollow needle according to claim 1, wherein the tip has at least one cutting edge and wherein the at least one cutting edge at the transition between the first section and the second section is aligned with the at least one cutting edge of the tip.

15. The hollow needle according to claim 1, wherein the tip comprises a first surface and a second surface which extend at an angle to the longitudinal axis of the inner tube over substantially half of the cross-section of the inner tube.

16. The hollow needle according to claim 15, wherein the two surfaces of the cutting edge are aligned along the longitudinal axis of the inner tube with the first surface and second surface of the tip.
